(19) United States
(12) Patent Application Publication
(10) Pub. No.: US 2001/0047123 A1
(43) Pub. Date: Nov. 29, 2001

(54) Method of manufacturing an endoscope component and an endoscope component manufactured by the method

(75) Inventors: Masanao Abe, Saitama (JP); Shinji Hayakawa, Saitama (JP)

Correspondence Address: GREENBLUM & BERNSTEIN, P.L.C., 1941 ROLAND CLARKE PLACE, RESTON, VA, 20191, US

(73) Assignee: ASAHI KOGAKU KOGYO KABUSHIKI KAISHA, Tokyo (JP)

(21) Appl. No.: 09/793,487

(22) Filed: Feb. 27, 2001

(30) Foreign Application Priority Data
Feb. 28, 2000 (JP) 2000-52068

(51) Int. Cl. A61B 1/00; C25D 11/24; C25D 11/18

(52) U.S. Cl. 600/101; 205/203; 205/202

(57) ABSTRACT

A method of manufacturing an endoscope component comprises the steps of preparing a base body made of aluminum or aluminum alloy; subjecting the base body to anodizing to form porous oxide coating on the surface of the base body; subjecting the base body to primary sealing treatment after the anodizing to seal at least a part of each of pores of the porous oxide coating; and subjecting the base body to secondary sealing treatment after the primary sealing treatment to seal each of the pores of the porous oxide coating (almost completely). In this method, the primary sealing treatment is carried out according to a metallic salt sealing process or a boiling water sealing process. Further, the secondary sealing treatment is carried out according to a steam sealing process. According to the method described above, it is possible to provide an endoscope component having excellent chemical resistance.

### BACKGROUND OF THE INVENTION

1. Field of the Invention

The present invention relates to a method of manufacturing an endoscope component and an endoscope component manufactured by the method.

2. Description of the Prior Art

In the medical field, an endoscope is used when examining the stomach, duodenum, small intestine, large intestine, or the like. The endoscope is constructed from various components, and some endoscope components of the endoscope are formed of metal materials such as aluminum alloy, stainless steel and the like. (Hereinafter, a component for an endoscope that is formed of metal material is referred to as “metallic endoscope component” or simply “endoscope component.”) Among these materials, aluminum alloy is mainly used in order to reduce the weight of the endoscope.

In actual endoscopic examination, an insertion section of the endoscope is inserted deep into a body cavity of a living body. When the endoscope is inserted into the body cavity, a surface of the insertion section of the endoscope comes to contact with bacteria resident in the body cavity, so that bacteria adheres onto the surface of the insertion section. The bacteria adhered onto the insertion section has to be removed in order to make it possible to use the endoscope repeatedly for many patients. Therefore, the endoscope after use is normally subjected to chemical treatment such as sterilization treatment, cleaning treatment, and the like. Such chemical treatment is carried out by immersing the endoscope after use in a treatment solution such as antiseptic solution and the like. When carrying out the chemical treatment, some of the endoscope components come into contact with the treatment solution. Therefore, if the metallic endoscope components have no chemical resistance, such metallic endoscope components will corrode through the repeated chemical treatments.

In view of the fact described above, in the case where the metallic endoscope components are manufactured using, for example, aluminum alloy, each of the metallic endoscope components has been conventionally subjected to anodizing (anodic oxidation treatment) and steam sealing treatment in its manufacturing process in order to give chemical resistance thereto. By carrying out such anodizing, a porous oxide coating is formed on a base body (which is used as a base for a metallic endoscope component). Further, by carrying out the steam sealing treatment, pores of the porous oxide coating formed on the base body are sealed. As a result of the anodizing and the sealing treatment, chemical resistance to the treatment solution as described above is given to the metallic endoscope components.

However, when a metallic endoscope component is manufactured according to the conventional manner described above, there is a possibility that the finally obtained endoscope cannot exhibit sufficient chemical resistance. For example, in the case where the endoscope is immersed in the antiseptic solution for a long time, or in the case where the endoscope is immersed in high concentration hydrogen peroxide type disinfecting solution (which is widely used in recent years), there is a possibility that the endoscope component changes in its color, appearance and quality. Further, in such a case, there is a possibility that corrosion develops on the endoscope components. This results in a problem that, for example, disinfecting solution and the like invade a watertight section of the endoscope, and thus the functions of the endoscope is deteriorated. In addition, when corrosion has developed on the endoscope components, bacteria and the like easily multiply at an area where the corrosion has developed, and this causes a problem of insanitary condition of the endoscope.

### SUMMARY OF THE INVENTION

In view of the above, it is an object of the present invention to provide a method of manufacturing an endoscope component having excellent chemical resistance.

Further, it is another object of the present invention to provide an endoscope component having excellent chemical resistance.

In order to achieve the above objects, the present invention is directed to a method of manufacturing an endoscope component which is used as one of components for an endoscope, comprising the steps of:

preparing a base body which is used as a base of the endoscope component, the base body being made of aluminum or aluminum alloy;

subjecting the base body to anodizing to form porous oxide coating with pores on a surface of the base body;

subjecting the base body to primary sealing treatment after the anodizing to seal at least a part of each of the pores of the porous oxide coating; and

subjecting the base body to secondary sealing treatment after the primary sealing treatment to seal each of the pores of the porous oxide coating thereby forming the endoscope component, the secondary sealing treatment being carried out in accordance with a steam sealing process.

According to the present invention as described in the above, it is possible to provide an endoscope component which has excellent chemical resistance, and in which any changes such as corrosion and the like do not occur even when an endoscope having the endoscope component repeatedly undergoes chemical treatments (such as disinfecting treatment, sterilizing treatment, cleaning treatment, and the like).

In this invention, it is preferred that the porous oxide coating has a thickness of 1 to 45 μm.

Further, in this invention, it is also preferred that the method further comprises, before the anodizing step, the step of subjecting the base body to cleaning treatment for at least one time.

Furthermore, in this invention, it is also preferred that the primary sealing treatment is carried out in accordance with a metallic salt sealing process or a boiling water sealing process. In the case of the metallic salt sealing process, it is preferred that the sealing treatment is carried out by immersing the base body in aqueous solution of metallic salt, in which the metallic salt in the aqueous solution contains at least one element selected from the group consisting of nickel, cobalt, cadmium, zinc, copper, aluminum, and lead. Further, in the case of the boiling water sealing process, it is preferred that the sealing treatment is carried out for 1-40 min using water held at 80 to 100° C.

Moreover, in this invention, it is also preferred that the method further comprises the step of subjecting the base body to coloring treatment before the primary sealing treatment. In this case, it is preferred that the coloring treatment is carried out in accordance with an electrolytic coloring process.

Still further, in this invention, it is also preferred that in the steam sealing process, the secondary sealing treatment is carried out for 5 to 45 min under the condition of a vapor pressure of 1.5 to 7 kg/cm².

Another aspect of the present invention is directed to an endoscope component which is used as one of components for an endoscope, wherein the endoscope component is manufactured according to the method of the present invention described above.

These and other objects, structures and advantages of the present invention will be apparent more clearly from the following description of the invention based on the examples.

### BRIEF DESCRIPTION OF THE DRAWINGS

FIG. 1 is generally shows an overall structure of a fiberscope which is an optical type endoscope;

FIG. 2 is generally shows an overall structure of an electronic scope which is an electronic type endoscope;

FIG. 3 is a partial sectional view which shows operating knobs of the fiberscope in FIG. 1 (or operating knobs of the electronic scope in FIG. 2);

FIG. 4 is a partial sectional view which shows an eyepiece section of the fiberscope in FIG. 1;

FIG. 5 is a process diagram which shows a manufacturing method of an endoscope component according to the present invention;

FIG. 6 is a sectional view which shows porous oxide coating (after primary sealing treatment) formed on a base body used as a base for the endoscope component; and

FIG. 7 is a sectional view which shows the porous oxide coating (after secondary sealing treatment) formed on the base body.

### DETAILED DESCRIPTION OF THE PREFERRED EMBODIMENTS

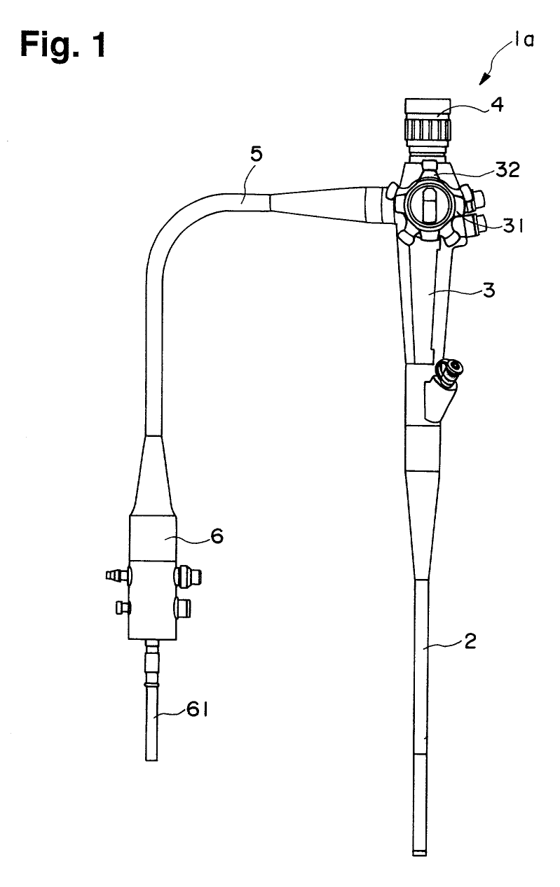

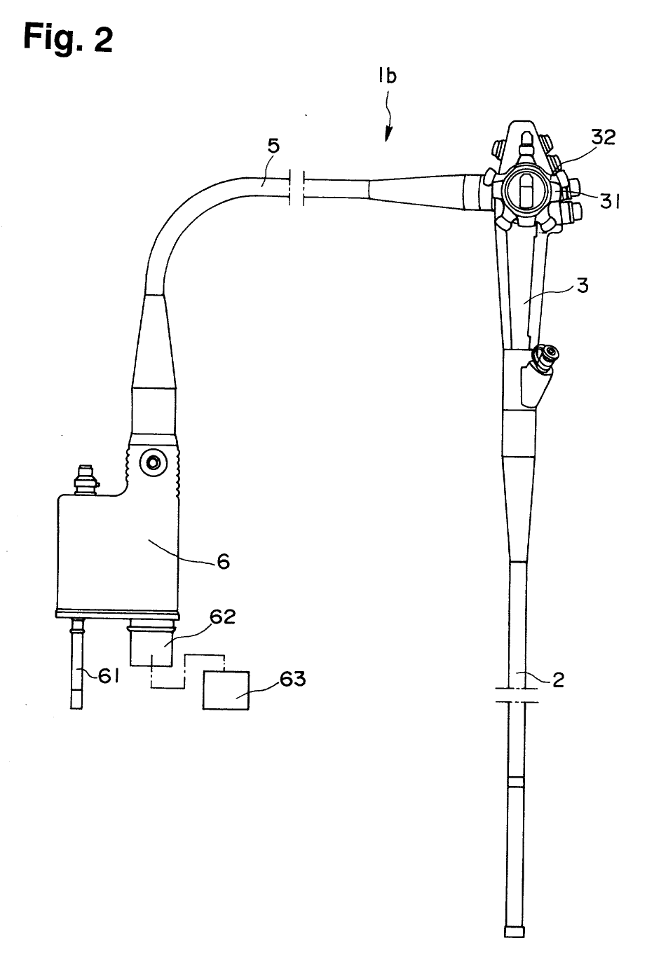

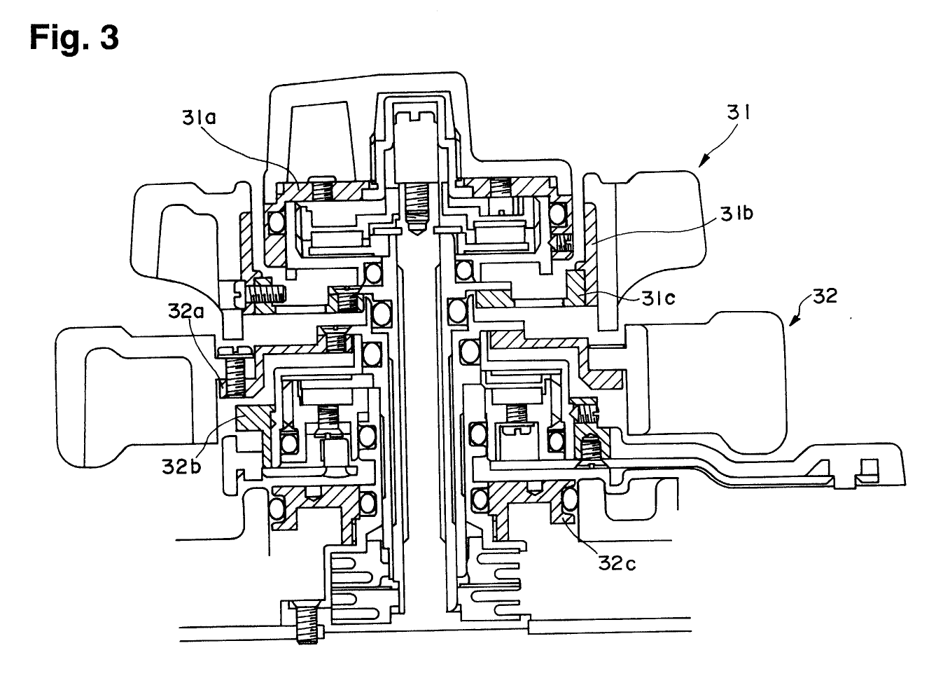

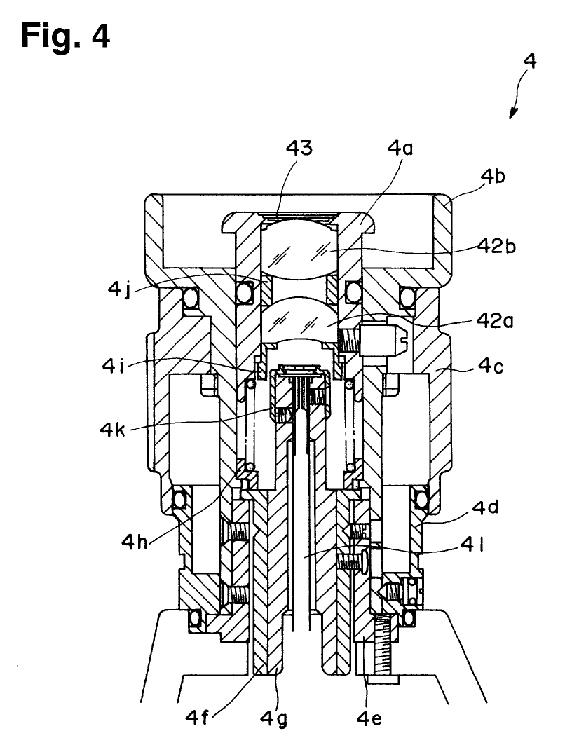

Hereinafter, a method of manufacturing an endoscope component and an endoscope component manufactured by the method according to the present invention will be described with reference to the appended drawings. FIG. 1 is generally shows an overall structure of a fiberscope which is an optical type endoscope. FIG. 2 is generally shows an overall structure of an electronic scope which is an electronic type endoscope. FIG. 3 is a partial sectional view which shows operating knobs of the fiberscope in FIG. 1 (or operating knobs of the electronic scope in FIG. 2). FIG. 4 is a partial sectional view which shows an eyepiece section of the fiberscope in FIG. 1. In the following descriptions, the upper side and the lower side in FIGS. 1, 2 and 4 will be referred to as a “base” and a “tip”, respectively.

As shown in FIG. 1, a fiberscope 1a has an insertion section (elongated flexible tube) 2 designed to be inserted into a body cavity of a living body; an operating section 3 provided on the base end of the insertion section 2, which is gripped by an operator during an endoscopic examination to manipulate the insertion section 2 of the fiberscope 1a; an eyepiece section 4 through which the operator observes an image of an object (e.g., an observation area of a patient), the eyepiece section 2 being provided on the base end of the operating section 3; a light guide flexible tube 5 connected to the operating section 3; and a plug 6 provided on the tip end of the light guide flexible tube 5.

On the side faces of the operating section 3, there are provided a right-and-left (RL) direction operating knob 31 and an up-and-down (UD) direction operating knob 32. When changing the direction of the insertion section 2 of the fiberscope 1a during the endoscopic examination, the operator turns each of the operating knobs 31 and 32 to pull appropriately wires (not shown) arranged in the insertion section 2. In this way, the insertion section 2 is bent to a desired direction.

As shown in FIG. 3, each of the operating knobs 31 and 32 includes a plurality of endoscope components. In these components, a ring-shaped RL locking member 31a, an RL outer ring member 31b, an RL inner ring member 31c, a UD ring member 32a, a ring-shaped UD locking member 32b, a cover 32c (which are shown with hatches in FIG. 3), and the like are made of aluminum or an aluminum alloy, and these metallic endoscope components are manufactured according to the method of this invention.

As shown in FIG. 1, on the tip end of the plug 6, there is provided a connector 61 which is connected to a light source device (not shown in the drawings). Light emitted from the light source device is guided to the tip end of the insertion section 2 through a light guide (not shown) formed from an optical fiber bundle. (The light guide extends inside the connector 61, the plug 6, the light guide flexible tube 5, the operating section 3, and the insertion section 2.) Then, the light is radiated from the tip end of the insertion section 2 to illuminate the observation area. Reflected light (which forms an image of the observation area) from the observation area illuminated by the light passes through an image guide (not shown in the drawings) formed from an optical fiber bundle. (The image guide extends inside the flexible tube 2 and the operating section 3.) Then, the reflected light is transmitted to the eyepiece section 4.

A base end portion 41 of the image guide can be seen in the central part of FIG. 4. As shown in this figure, the base end portion 41 is positioned at the central part of the eyepiece section 4. Above the end (upper side in FIG. 4) of the base end portion 41 of the image guide, there are provided an eyepiece frame 4a having roughly cylindrical shape and eyepieces 42a and 42b which are fixed inside the eyepiece frame 4a at predetermined positions. By constructing the fiberscope 1a as described above, the operator can observe an image formed by the light from the observation area through the eyepiece section 4.

At the base end of the eyepiece frame 4a, an annular stepped portion 43 is formed. By forming such an annular stepped portion, it becomes possible to reliably prevent the light from reflecting at the base end of the eyepiece frame 4a. This makes it possible for the operator to observe an image of the observation area more clearly.

In the components constituting the eyepiece section 4, the eyepiece frame 4a, an eyepiece cylinder 4b, an eyesight ring member 4c, a covering member 4d, an eyepiece axis member 4e, a centering member 4f, an image guide fixing member 4g, a spring holder 4h, a lens fixing member 4i, a spacer 4j, a cover glass fixing member 4k (which are shown with hatching in FIG. 4) and the like are formed of aluminum or an aluminum alloy, and these metallic endoscope components are manufactured according to the method of this invention.

Next, an electronic scope 1b shown in FIG. 2 will be described.

As will be described in the following, the electronic scope 1b has a structure substantially the same as that of the fiberscope 1a shown in FIG. 1 except that no eyepiece is provided and means for observing an observation area is different from that of the fiberscope 1a in FIG. 1.

The electronic scope 1b has no image guide. Instead of such an image guide, the electronic scope 1b has an imaging element (charge coupled device) for taking an image of an observation area. The imaging element is provided at the tip end of the insertion section 2. Further, as shown in FIG. 2, the electronic scope 1b has a connector 62 provided at the tip end of a plug 6. The connector 62 is connected to a light source device which is connected to a monitor (not shown) via a cable.

In the electronic scope 1b, the reflected light (which forms an image of the observation area) from the observation area is received by the imaging element. Then, the imaging element outputs an image signal corresponding to the image formed on the imaging element by the reflected light. The image signal is transmitted to the plug 6 via an image signal cable (not shown in the drawing) which extends inside the insertion section 2, an operating section 3 and a light guide flexible tube 5. Then, in the plug 6 and the light source device, the image signal is subjected to predetermined processing (such as signal processing, image processing, and the like), and then the processed signal is send to the monitor. In this way, an image (electronic image) taken by the imaging element is displayed on the screen of the monitor.

When cleaning and disinfecting the electronic scope 1b, a waterproofing cap 63 is attached to the connector 62 in order to protect the connector 62 from a chemical solution such as cleaning solution and disinfecting solution. The waterproofing cap 63 is formed of aluminum or aluminum alloy, and therefore this waterproofing cap 63 can also be manufactured according to the method of this invention.

Next, the method of manufacturing an endoscope component according to the present invention will be described.

In this invention, each of the endoscope components made of aluminum or aluminum alloy described above is generally manufactured according to the following steps (a)-(f).

(a) A step of preparing a base body which is used as a base of an endoscope component. (The base body is made of aluminum or aluminum alloy.)

(b) A step of subjecting the base body to alkali cleaning treatment (degreasing treatment).

(c) A step of subjecting the base body to anodizing (anodic oxidation treatment) to form porous oxide coating with pores on the surface of the base body.

(d) A step of subjecting the base body to coloring treatment after the anodizing to color the base body.

(e) A step of subjecting the base body to primary sealing (preliminary sealing) treatment after the anodizing to seal at least a part of each of the pores of the porous oxide coating.

(f) A step of subjecting the base body to secondary sealing (main sealing) treatment after the primary sealing to roughly completely (sufficiently) seal each of the pores of the porous oxide coating, thereby forming the endoscope component. (In this step, the secondary sealing is carried out in accordance with a steam sealing process.)

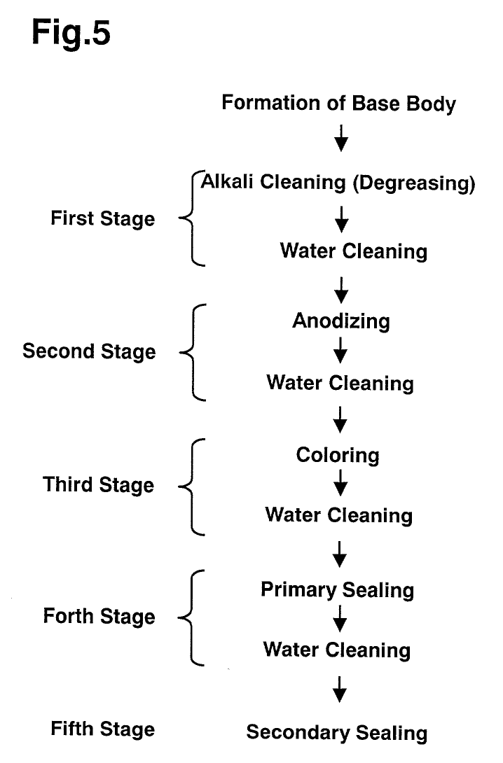

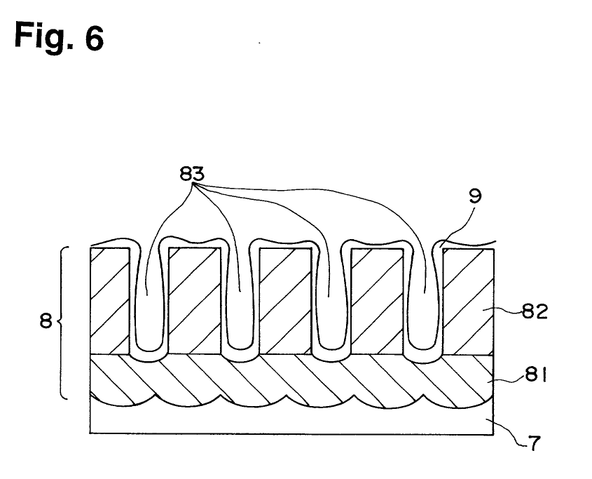

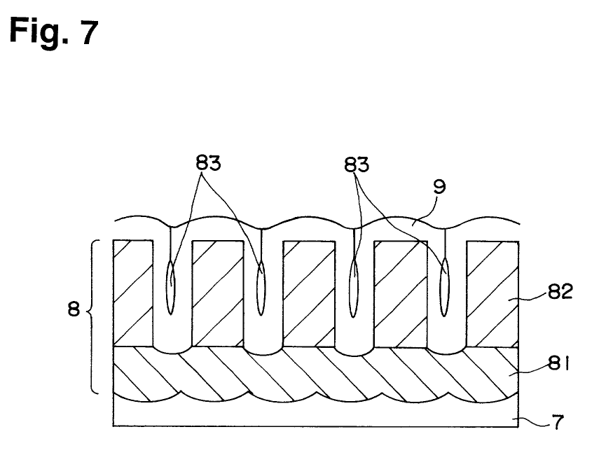

Hereinafter, the method of this invention will be described in more detail with reference to FIGS. 5-7. FIG. 5 is a process diagram which shows the manufacturing method of an endoscope component according to the present invention. FIG. 6 is a sectional view which shows porous oxide coating (after the primary sealing) formed on a base body. FIG. 7 is a sectional view which shows the porous oxide coating (after the secondary sealing) formed on the base body.

When manufacturing an endoscope component made of aluminum or aluminum alloy, first, a base body used as a base of an endoscope component is prepared by forming aluminum material or aluminum alloy material into a predetermined shape. Then, the base body is subjected to chemical treatments in the following five stages, which are also shown in FIG. 5.

(First Stage)

In the first stage, the base body is subjected to alkali cleaning treatment (degreasing treatment), and then it is subjected to water cleaning. Through the alkali cleaning, the surface of the base body is dissolved slightly. By carrying out such alkali cleaning, it is possible to remove from the base body various kinds of fat soluble substances attached to the surface of the base body, as well as impurities embedded in the vicinity of the surface of the base body. As a result of the alkali cleaning, it becomes possible to more uniformly and homogeneously form porous oxide coating on the basic body in the anodizing in the next stage.

Alkali solution to be used for the alkaline cleaning in this stage is not particularly limited. Examples of such alkali solution include aqueous solution of sodium hydroxide, aqueous solution of potassium hydroxide, and the like.

(Second Stage)

In the second stage, the base body is subjected to the anodizing, and then it is subjected to water cleaning. In the anodizing, first the base body as an anode is immersed in an acidic aqueous solution bath, and then electric current is applied thereto. Examples of acid used in the acidic aqueous solution include sulfuric acid, oxalic acid, chromic acid, phosphoric acid, formic acid, nitric acid, sulfamic acid, malonic acid, aconitic acid, maleic acid, citraconic acid, itaconic acid, and the like. In this invention, it is preferable to use the aqueous solution of the sulfuric acid or oxalic acid.

As a result of the anodizing, porous oxide coating 8 having pores 83 is formed on the surface of the base body 7 as shown in FIG. 6. The porous oxide coating 8 has a duplex-layer structure which includes a dense barrier layer 81 formed on the base body 7, and a porous layer 82 formed on the barrier layer 81.

It is to be noted that in the case where a neutral aqueous solution is used in the anodizing instead of the acidic aqueous solution, only an oxide film like the barrier layer 81 is formed on the base body 7 without formation of the porous layer 82. In this case, it is difficult to give a large thickness to the oxide film, and therefore there is a possibility that a finally obtained endoscope component cannot exhibit sufficient chemical resistance.

In the anodizing, the content of acid in the acidic aqueous solution (that is, the acid concentration) is preferably about 1 to 50 wt %. The amount of dissolved aluminum in the acidic aqueous solution is preferably about 1 to 7 g/L. The applied voltage in the anodizing is preferably about 5 to 150V. The current density in the anodizing is preferably about 20 to 900 A/m². In this case, the current to be applied may be either DC or AC. The temperature of the acidic aqueous solution bath in the anodizing is preferably about 10 to 50° C. The anodizing time is preferably about 3 to 90 min. When the respective conditions for the anodizing are set to the above ranges, it is possible to form the porous oxide film 8 on the base body 7 more uniformly and more homogeneously.

The thickness of the porous oxide coating 8 to be formed on the surface of the base body 7 is not particularly limited. The thickness of the porous oxide coating 8 is preferably about 1 to 45 μm, and more preferably 5 to 30 μm. In the case where the thickness of the porous oxide coating 8 is less than the lower limit of the above range, there is a possibility that a finally obtained endoscope component cannot exhibit sufficient chemical resistance. On the other hand, even if the thickness larger than the upper limit of the above range is given to the porous oxide coating 8, it is impossible to obtain an endoscope component having higher chemical resistance, and rather this is disadvantageous in miniaturizing an endoscope component.

(Third Stage)

In the third stage, the base body is subjected to coloring treatment, and then it is subjected to water cleaning. This stage may be omitted, if unnecessary.

A process for coloring the base body is not particularly limited. Examples of such a process include an electrolytic coloring process, a natural coloring process, and the like. Among these processes, the electrolytic coloring process is preferably used in this invention. As a result of this process, the surface of the endoscope component is colored in, for example, blackish brown or black. For example, by coloring the eyepiece frame 4a (which is one of metallic endoscope components) through the electrolytic coloring, it becomes possible to prevent the reflected light from the observation area from reflecting at the annular stepped portion 43 of the eyepiece frame 4a, more reliably. As a result, the operator can observe the image of the observation area more clearly through the eyepiece section 4.

The electrolytic coloring is carried out by immersing the base body in an electrolytic bath and then applying electric current thereto, whereby metal such as nickel and cobalt is deposited on the surface of the porous oxide coating 8 on the base body 7. Examples of an aqueous solution in the electrolytic bath include an aqueous solution in which boric acid and sulfate are dissolved. Further, examples of such sulfate include nickel sulfate, ammonium nickel sulfate, cobalt sulfate, and the like. In this case, the content of the sulfate in the aqueous solution (that is, the sulfate concentration) is preferably about 10 to 50 g/L. Further, the content of the boric acid in the aqueous solution (that is, the concentration of boric acid) is preferably about 10 to 40 g/L.

When the cobalt sulfate is used, an aqueous solution in which ammonium sulfate as well as the cobalt sulfate is dissolved is used for the electrolytic bath. In this case, the content of the ammonium sulfate in the aqueous solution (that is, the concentration of the ammonium sulfate) is preferably about 5 to 30 g/L.

In the electrolytic coloring, the applied voltage is preferably about 5 to 30V. The current density is preferably about 0.05 to 5.0 A/dm². T he pH of the aqueous solution is preferably about 1.9 to 6.1. The temperature of the electrolytic bath is preferably about 5 to 30° C. Further, the electrolyzing time is preferably about 3 to 20 min. When the respective conditions for the electrolytic coloring are set to the above ranges, it is possible to deposit metal (such as nickel, cobalt and the like) on the surface of the porous oxide coating 8 more effectively. As a result, the surface of the endoscope component is properly colored.

(Fourth Stage)

In the fourth stage, the base body is subjected to the primary sealing treatment, and then it is subjected to water cleaning. Through the primary sealing, thin coating (sealing film) 9 is formed on the surface of the porous oxide coating 8 as shown in FIG. 6. In other wards, through the primary sealing, at least a part of each of the pores 83 is preliminarily sealed. By carrying out such primary sealing as mentioned above, it becomes possible to reliably prevent stains from generating on the surface of the base body after the coloring treatment, for example. Further, it also becomes possible to prevent the metal deposited in the coloring treatment from peeling off. Accordingly, by carrying out the primary sealing treatment onto the base body, it becomes possible to effectively prevent the appearance of a finally obtained endoscope component from being changed.

A process of achieving the primary sealing is not particularly limited. In this invention, however, it is preferable that such primary sealing is carried out according to a sealing process such as a metallic salt sealing process, a boiling water sealing process, and the like.

In the case where the primary sealing is carried out according to the metallic salt sealing process, conditions for the treatment are not particularly limited. In this invention, however, it is preferred that the metallic salt sealing is carried out by immersing the base body in an aqueous solution of metallic salt. In this case, it is preferable that the metallic salt in the aqueous solution contains at least one element selected from the group consisting of nickel, cobalt, cadmium, zinc, copper, aluminum, and lead. These metal elements maybe in the form of acetate, nitrate or sulfate. The content of the metallic salt in the aqueous solution (that is, the concentration of the metallic salt) is not particularly limited, but the metallic salt content is preferably about 4.0 to 7.0 g/L, and more preferably about 5.0 to 5.8 g/L. The aqueous solution preferably has a pH of about 3.5 to 6.9, and more preferably a pH of about 4.9 to 6.1. The temperature of the aqueous solution in the metallic salt sealing preferably lies within the range of about 15 to 100° C., and more preferably about 50 to 90° C. The sealing time is preferably about 1 to 8 min, and more preferably about 3 to 6 min. When conditions for the metallic salt sealing are respectively set to the ranges described above, it becomes possible to carry out the primary sealing treatment more effectively.

On the other hand, in the case of the boiling water sealing process, the primary sealing is carried out by immersing the base body in boiling water. In this invention, water to be used in the boiling water sealing is not particularly limited, but it is preferable to use distilled water, ion exchange water, RO (Reverse Osmosis) water, or the like. When such water is used, it becomes possible to prevent proper formation of the sealing film 9 from being hindered by impurities such as chloride ion. In the boiling water sealing, the sealing temperature is not particularly limited, but it is preferably about 80 to 100° C., and more preferably about 90 to 100° C. The sealing time is not particularly limited, but it is preferably about 1 to 40 min, and more preferably about 15 to 30 min. When the boiling water sealing is carried out according to the conditions set to the above ranges, it is possible to carry out the primary sealing more effectively.

(Fifth Stage)

In the fifth stage, the base body is subjected to the secondary sealing (main sealing) treatment. In the secondary sealing, the thickness of the sealing film 9 formed through the primary sealing is increased, and finally the openings 83 of the porous oxide film 8 are almost completely sealed as shown in FIG. 7. In this invention, the secondary sealing is carried out in accordance with a steam sealing process.

In this invention, conditions for the steam sealing are set to the ranges described below. Namely, the steam pressure is not particularly limited, but it is preferably about 1.5 to 7 kg/cm², and more preferably about 2 to 5 kg/cm². The sealing time is not particularly limited, but it is preferably about 5 to 45 min, and more preferably about 10 to 30 min. In the case where the respective conditions for the secondary sealing treatment are below the lower limits of the ranges described above, there is a possibility that it is impossible to properly carry out the secondary sealing treatment. On the other hand, even in the case where the respective conditions are over the upper limits of the ranges described above, it is impossible to obtain an endoscope component having higher chemical resistance.

In this regard, it is to be understood that in the case where this sealing treatment is carried out in a single stage without the preliminary sealing treatment as described above with reference to the fourth stage, there is a possibility that the pores 83 of the porous oxide coating 8 are not sufficiently sealed. As a result, an endoscope component manufactured through such a single stage sealing treatment involves a problem in that the endoscope component cannot exhibit sufficient chemical resistance when it is immersed in, for example, a hydrogen peroxide type disinfecting solution having high concentration. In contrast with this, according to the manufacturing method of this invention, sealing treatment is carried out in two stages (i.e., the preliminary sealing stage and the main sealing stage). By carrying out the sealing treatment in such two stages as described above, it becomes possible to more densely and sufficiently seal the pores 83 of the porous oxide coating 8 on the base body. As a result, even when immersed in a high concentration disinfecting solution as described above, the endoscope component manufactured according to the present invention can exhibit excellent chemical resistance.

Through the above-mentioned five stages, the porous oxide coating 8 is formed on the surface of the base body made of aluminum or aluminum alloy, the sealing film 9 is formed on the porous oxide coating 8, and then the pores 83 of the porous oxide coating 8 are sufficiently (almost completely) sealed.

In this invention, it is preferable that the water cleaning in each of the first—fourth stages is carried out using distilled water, ion exchange water, RO (Resistance Osmosis) water or the like.

In the above, the method of manufacturing an endoscope component according to the present invention was described. In this connection, it is to be noted that this invention is not limited to the embodiments described above. For example, in the present invention, the water cleaning in each or any one of the stages may be omitted, if unnecessary.

### EXAMPLES

Next, specific examples of the present invention will be described below.

### Example 1a

First, a base body was prepared by forming an aluminum material (which is classified in JIS H4000 with number of more than A5000 but less than A6000) into a predetermined shape. The prepared base body was subjected to the following treatments (1)-(5).

(1) First, alkaline cleaning was carried out to the base body for 5 min using aqueous solution of sodium hydroxide held at 50° C. Then, water cleaning was carried out using distilled water.

(2) Next, anodizing was carried out to form porous oxide coating with thickness of 20 μm on the surface of the base body, and then water cleaning was carried out using distilled water. This anodizing was carried out under the following conditions using an aqueous solution of sulfuric acid.

Content of Sulfuric Acid in Aqueous Solution: 20 wt %

Content of Dissolved Aluminum in Aqueous Solution: 2.5 g/L

Temperature of Aqueous Solution: 20° C.

Voltage: 20V

Current Density: 100 A/m²

Type of Electrolysis: DC Electrolysis

Anodizing Time: 30 min

(3) Next, coloring treatment was carried out to the base body according to an electrolytic coloring process to color the surface of the base body in black, and then water cleaning was carried out using distilled water. The electrolytic coloring was carried out under the following conditions.

Content of Cobalt Sulfate in Electrolytic Bath: 20 g/L

Content of Boric Acid in Electrolytic Bath: 25 g/L

Content of Ammonium Sulfate in Electrolytic Bath: 15 g/L

pH of Aqueous solution in Electrolytic Bath: 4

Temperature of Aqueous solution in Electrolytic Bath: 20° C.

Voltage: 15V

Current Density: 0.3 A/dm²

Coloring Time: 10 min

(4) Next, primary sealing (preliminary sealing) treatment was carried out to the base body, and then water cleaning was carried out using distilled water. The primary sealing was carried out under the following conditions according to a metallic salt sealing process.

Content of Nickel Acetate in Aqueous Solution: 5.5 g/L

pH of Aqueous Solution: 5.5

Temperature of Aqueous Solution: 70° C.

Sealing Time: 3 min

(5) Next, secondary sealing (main sealing) treatment was carried out to the base body under the following conditions according to a steam sealing process.

Steam Pressure: 5 kg/cm²

Sealing Time: 15 min

According to the treatments (1)-(5) described above, endoscope components used for an endoscope were manufactured. Then, four endoscopes were manufactured using the manufactured endoscope components.

### Example 1b

Endoscope components were manufactured according to the same manner as in Example 1a except that instead of nickel acetate, lead acetate was used as metallic salt in an aqueous solution for the primary sealing. Then, four endoscopes were manufactured using the manufactured endoscope components.

### Example 1c

Endoscope components were manufactured according to the same manner as in Example 1a except that instead of nickel acetate, cobalt acetate was used as metallic salt in an aqueous solution for the primary sealing. Then, four endoscopes were manufactured using the manufactured endoscope components.

### Example 1d

Endoscope components were manufactured according to the same manner as in Example 1a except that the primary sealing treatment was carried out according to a boiling water sealing process. Then, four endoscopes were manufactured using the manufactured endoscope components. In this Example, the primary sealing was carried out under the following conditions using distilled water.

Temperature of Water: 95° C.

Sealing Time: 20 min

### Comparative Example 1

Endoscope components were manufactured according to the same manner as in Example 1a except that the primary sealing process (that is, the process (4) in Example 1a) was omitted. Then, four endoscopes were manufactured using the manufactured endoscope components.

(Evaluation)

Four endoscopes manufactured in each of Examples 1a-1d and Comparative Example 1 were subjected to the following disinfecting treatments (i)-(iv), respectively.

(i) Disinfecting treatment in which each endoscope was immersed in an aqueous solution of hydrogen peroxide held at 20° C. for 60 min. (The content of the hydrogen peroxide in the aqueous solution was 12 wt %.) This treatment was repeated 300 times for each endoscope.

(ii) Disinfecting treatment in which each endoscope was immersed in an aqueous solution of peracetic acid held at 55° C. for 60 min. (The content of the peracetic acid in the aqueous solution was 0.8 wt %.) This treatment was repeated 300 times for each endoscope.

(iii) Disinfecting treatment in which each endoscope was immersed in an aqueous solution of glutaraldehyde held at 60° C. for 60 min. (The content of the glutaraldehyde in the aqueous solution was 0.5 wt %.) This treatment was repeated 300 times for each endoscope.

(iv) Disinfecting treatment in which ethylene oxide gas (EOG) was applied to each endoscope for 60 min. This treatment was repeated 300 times for each endoscope.

During and after each of the disinfecting treatments (i)-(iv), the changes in the appearance of an eyepiece flame (which is one of the endoscope components made of aluminium, and is shown in FIG. 4 with the reference numeral “4a”) were observed for each endoscope. The appearance changes were evaluated in accordance with the four rankings A-D given below.

Ranking A: No change in the appearance was observed on the eyepiece frame even after the completion of 300 times.

Ranking B: Between 200 and 299 times, color fade-out and corrosion were observed on the annular stepped portion of the eyepiece frame.

Ranking C: Between 100 and 199 times, color fade-out and corrosion were observed on the annular stepped portion of the eyepiece frame.

Ranking D: Between 1 and 99 times, color fade-out and corrosion were observed on the annular stepped portion of the eyepiece frame.

The evaluation results are shown in the attached Table 1.

### Example 2a

First, a base body was prepared by forming an aluminum material (which is classified in JIS H4000 with number of more than A5000 but less than A6000) into a predetermined body. The prepared base body was subjected to the following treatments (1)-(4).

(1) First, alkaline cleaning was carried out to the base body for 5 min using an aqueous solution of sodium hydroxide held at 50° C. Then, water cleaning was carried out using distilled water.

(2) Next, anodizing was carried out to form porous oxide coating with thickness of 20 μm on the surface of the base body, and then water cleaning was carried out using distilled water. This anodizing was carried out under the following conditions using an aqueous solution of oxalic acid.

Content of Oxalic Acid in Aqueous Solution: 5 wt %

Content of Dissolved Aluminum in Aqueous Solution: 1.5 g/L

Temperature of Aqueous Solution: 30° C.

Voltage: 40V

Current Density: 100 A/m²

Type of Electrolysis: DC Electrolysis

Anodizing Time: 60 min

(3) Next, primary sealing (preliminary sealing) treatment was carried out to the base body, and then water cleaning was carried out using distilled water. The primary sealing was carried out under the following conditions according to a metallic salt sealing process.

Content of Nickel Acetate in Aqueous Solution: 5.5 g/L

pH of Aqueous Solution: 5.5

Temperature of Aqueous Solution: 70° C.

Sealing Time: 3 min

(4) Next, secondary sealing (main sealing) treatment was carried out to the base body under the following conditions according to a steam sealing process.

Steam Pressure: 5 kg/cm²

Sealing Time: 15 min

According to the treatments (1)-(4) describedabove, endoscope components used for an endoscope were manufactured. Then, four endoscopes were manufactured using the manufactured endoscope components.

### Example 2b

Endoscope components were manufactured according to the same manner as in Example 2a except that instead of nickel acetate, lead acetate was used as metallic salt in an aqueous solution for the primary sealing. Then, four endoscopes were manufactured using the manufactured endoscope components.

### Example 2c

Endoscope components were manufactured according to the same manner as in Example 2a except that instead of nickel acetate, cobalt acetate was used as metallic salt in an aqueous solution for the primary sealing. Then, four endoscopes were manufactured using the manufactured endoscope components.

### Example 2d

Endoscope components were manufactured according to the same manner as in Example 2a except that the primary sealing treatment was carried out according to a boiling water sealing process. Then, four endoscopes were manufactured using the manufactured endoscope components. In this Example, the primary sealing was carried out under the following conditions using distilled water.

Temperature of Water: 95° C.

Sealing Time: 20 min

### Comparative Example 2

Endoscope components were manufactured according to the same manner as in Example 2a except that the primary sealing process (that is, the process (3) in Example 2a) was omitted. Then, four endoscopes were manufactured using the manufactured endoscope components.

(Evaluation)

Four endoscopes manufactured in each of Examples 2a-2d and Comparative Example 2 were subjected to the following disinfecting treatments (i)- (iv), respectively.

(i) Disinfecting treatment in which each endoscope was immersed in an aqueous solution of hydrogen peroxide held at 20° C. for 60 min. (The content of the hydrogen peroxide in the aqueous solution was 12 wt %.) This treatment was repeated 300 times for each endoscope.

(ii) Disinfecting treatment in which each endoscope was immersed in an aqueous solution of peracetic acid held at 55° C. for 60 min. (The content of the peracetic acid in the aqueous solution was 0.8 wt %.) This treatment was repeated 300 times for each endoscope.

(iii) Disinfecting treatment in which each endoscope was immersed in an aqueous solution of glutaraldehyde held at 60° C. for 60 min. (The content of the glutaraldehyde in the aqueous solution was 0.5 wt %.) This treatment was repeated 300 times for each endoscope.

(iv) Disinfecting treatment in which ethylene oxide gas (EOG) was applied to each endoscope for 60 min. This treatment was repeated 300 times for each endoscope.

During and after each of the disinfecting treatments (i)-(iv), the changes in the appearance of an RL inner ring member (which is one of the endoscope components made of aluminium, and is shown in FIG. 3 with the reference numeral “31c”) were observed for each endoscope. The appearance changes were evaluated in accordance with the four rankings A-D given below.

Ranking A: No corrosion was observed on the RL inner ring member even after the completion of 300 times.

Ranking B: Between 200 and 299 times, corrosion was observed on the RL inner ring member.

Ranking C: Between 100 and 199 times, corrosion was observed on the RL inner ring member.

Ranking D: Between 1 and 99 times, corrosion was observed on the RL inner ring member.

The evaluation results are shown in the attached Table 2.

(Evaluation Results)

The results in Tables 1 and 2 show that the endoscope component of each endoscope manufactured in Examples 1a-1d and 2a-2d can exhibit excellent chemical resistance to the aqueous solution of hydrogen peroxide, the aqueous solution of peracetic acid and the aqueous solution of glutaraldehyde as well as the ethylene oxide gas.

Further, the results in Tables 1 and 2 also show that the endoscope component (eyepiece flame 4) of each endoscope manufactured in Comparative Example 1 exhibits sufficient chemical resistance to the ethylene oxide gas, but it exhibits poor chemical resistance to the aqueous solution of hydrogen peroxide, the aqueous solution of peracetic acid, and the aqueous solution of glutaraldehyde. The endoscope having the endoscope components manufactured in Comparative Example 1 was actually used to observe an object. In this observation, light transmitted from the object was reflected at the annular stepped portion of the eyepiece flame (where color-fadeout (whitening) and corrosion had developed), and thus an operator could not observe an image of the object clearly.

Furthermore, the results in Tables 1 and 2 also show that the endoscope component (RL inner ring member 31c) of each endoscope manufactured in Comparative Example 2 exhibits sufficient chemical resistance to the ethylene oxide gas, but it exhibits poor chemical resistance to the aqueous solution of hydrogen peroxide, the aqueous solution of peracetic acid, and the aqueous solution of glutaraldehyde. The endoscope having the endoscope components manufactured in Comparative Example 2 was actually used to observe an object. In this observation, the operator could not smoothly operate operating knobs of the endoscope because of the RL inner ring member where corrosion had developed.

According to the present invention as described in the above, it is possible to provide an endoscope component which has excellent chemical resistance, and in which any changes such as corrosion and the like do not occur even when an endoscope having the endoscope component repeatedly undergoes chemical treatments (such as disinfecting treatment, sterilizing treatment, cleaning treatment, and the like).

Finally, it is to be understood that many changes and additions may be made to the embodiments described above without departing from the scope and spirit of the invention as defined in the appended claims.

Further, it is also to be understood that the present disclosure relates to subject matter contained in Japanese Patent Application No. 2000-052068 (filed on Feb. 28, 2000) which is expressly incorporated herein by reference in its entirety.

TABLE 1

| | | | | | Evaluation Results | | | |
|---|---|---|---|---|---|---|---|---|
| | | | | | Aqueous | Aqueous | | |
| | Aqueous | | | | Solution | Solution | | |
| | Solution | | | | of | of | Aqueous | |
| | Used in | | Primary | Secondary | Hydrogen | Peracetic | Solution of | Ethylene |
| | Anodizing | Coloring | Sealing | Sealing | Peroxide | Acid | Glutaraldehyde | Oxide Gas |
| Example 1a | Aqueous | Electrolytic | Metallic Salt | Steam Sealing | A | A | A | A |
| | Solution | Coloring | Sealing | | | | | |
| | of | | (Metallic Salt: | | | | | |
| | Sulfuric | | Nickel Acetate | | | | | |
| | Acid | | | | | | | |
| Example 1b | | | Metallic Salt | | A | A | A | A |
| | | | Sealing | | | | | |
| | | | (Metallic Salt: | | | | | |
| | | | Lead Acetate | | | | | |
| Example 1c | | | Metallic Salt | | A | A | A | A |
| | | | Sealing | | | | | |
| | | | (Metallic Salt: | | | | | |
| | | | Cobalt | | | | | |
| | | | Acetate | | | | | |
| Example 1d | | | Boiling Water | | A | A | A | A |
| | | | Sealing | | | | | |
| Comparative | Aqueous | Electrolytic | — | Steam Sealing | D | C | D | A |
| Example 1 | Solution | Coloring | | | | | | |
| | of | | | | | | | |
| | Sulfuric | | | | | | | |
| | Acid | | | | | | | |

TABLE 2

| | | | | | Evaluation Results | | | |
|---|---|---|---|---|---|---|---|---|
| | | | | | Aqueous | Aqueous | | |
| | Aqueous | | | | Solution | Solution | | |
| | Solution | | | | of | of | Aqueous | |
| | Used in | | Primary | Secondary | Hydrogen | Peracetic | Solution of | Ethylene |
| | Anodizing | Coloring | Sealing | Sealing | Peroxide | Acid | Glutaraldehyde | Oxide Gas |
| Example 2a | Aqueous | — | Metallic Salt | Steam Sealing | A | A | A | A |
| | Solution | | Sealing | | | | | |
| | of Oxalic | | (Metallic Salt: | | | | | |
| | Acid | | Nickel Acetate | | | | | |
| Example 2b | | | Metallic Salt | | A | A | A | A |
| | | | Sealing | | | | | |
| | | | (Metallic Salt: | | | | | |
| | | | Lead Acetate | | | | | |
| Example 2c | | | Metallic Salt | | A | A | A | A |
| | | | Sealing | | | | | |
| | | | (Metallic Salt: | | | | | |
| | | | Cobalt | | | | | |
| | | | Acetate | | | | | |
| Example 2d | | | Boiling Water | | A | A | A | A |
| | | | Sealing | | | | | |
| Comparative | Aqueous | — | — | Steam Sealing | D | C | D | A |
| Example 2 | Solution | | | | | | | |
| | of Oxalic | | | | | | | |
| | Acid | | | | | | | |

What is claimed is:

1. A method of manufacturing an endoscope component which is used as one of components for an endoscope, comprising the steps of:
preparing a base body which is used as a base of the endoscope component, the base body being made of aluminum or aluminum alloy;
subjecting the base body to anodizing to form porous oxide coating with pores on a surface of the base body;
subjecting the base body to primary sealing treatment after the anodizing to seal at least a part of each of the pores of the porous oxide coating; and
subjecting the base body to secondary sealing treatment after the primary sealing treatment to seal each of the pores of the porous oxide coating thereby forming the endoscope component, the secondary sealing treatment being carried out in accordance with a steam sealing process.

2. The method as claimed in claim 1, wherein the porous oxide coating has a thickness of 1 to 45 μm.

3. The method as claimed in claim 1, further comprising, before the anodizing step, the step of subjecting the base body to cleaning treatment for at least one time.

4. The method as claimed in claim 1, wherein the primary sealing treatment is carried out in accordance with a metallic salt sealing process or a boiling water sealing process.

5. The method as claimed in claim 4, wherein in the metallic salt sealing process, the sealing treatment is carried out by immersing the base body in aqueous solution of metallic salt, in which the metallic salt in the aqueous solution contains at least one element selected from the group consisting of nickel, cobalt, cadmium, zinc, copper, aluminum, and lead.

6. The method as claimed in claim 4, wherein in the boiling water sealing process, the sealing treatment is carried out for 1 to 40 min using water held at 80 to 100° C.

7. The method as claimed in claim 1, further comprising the step of subjecting the base body to coloring treatment before the primary sealing treatment.

8. The method as claimed in claim 7, wherein the coloring treatment is carried out in accordance with an electrolytic coloring process.

9. The method as claimed in claim 1, wherein in the steam sealing process, the secondary sealing treatment is carried out for 5 to 45 min under the condition of a vapor pressure of 1.5 to 7 kg/cm².

10. An endoscope component which is used as one of components for an endoscope, wherein the endoscope component is manufactured according to the method as claimed in any one of claims 1 to 9.
